(19) United States
(12) Patent Application Publication
(10) Pub. No.: US 2023/0113687 A1
(43) Pub. Date: Apr. 13, 2023

(54) SYSTEMS AND METHODS FOR ROBOTIC ENDOSCOPIC SUBMUCOSAL DISSECTION

(71) Applicant: Noah Medical Corporation, San Carlos, CA (US)

(72) Inventor: Enrique Romo, Redwood City, CA (US)

(21) Appl. No.: 18/054,810

(22) Filed: Nov. 11, 2022

Related U.S. Application Data
(63) Continuation of application No. PCT/US2021/035220, filed on Jun. 1, 2021.
(60) Provisional application No. 63/033,428, filed on Jun. 2, 2020.

(51) Int. Cl. A61B 1/008; A61B 1/05; A61B 1/005; A61B 1/12; A61B 1/00

(52) U.S. Cl. CPC A61B 1/008; A61B 1/05; A61B 1/0057; A61B 1/126; A61B 1/00009

(57) ABSTRACT

A robotic device is provided. The robotic device comprises: an articulatable elongate member comprising a proximal end and a distal end, and the distal end is steerable via a first driving mechanism; an articulatable imaging instrument removably coupled to the articulatable elongate member via a first lumen of the articulatable elongate member, and the articulatable imaging instrument comprises a camera located at a distal portion of the articulatable imaging instrument; an articulatable instrument removably coupled to the articulatable elongate member via a second lumen, and an operation of the articulatable instrument is captured by the camera of the articulatable imaging instrument.

### REFERENCE

This application is a Continuation Application of International Application No. PCT/US2021/035220, filed Jun. 1, 2021, which claims the benefit of U.S. Provisional Application No. 63/033,428, filed Jun. 2, 2020, which applications are incorporated herein by reference.

### BACKGROUND OF THE INVENTION

Gastric and colorectal cancers are the common types of cancer. Gastrointestinal (GI) cancers grow from the mucosal layer. Survival rates for patients suffering from these cancers may be improved if pre-malignant and early cancers are removed at an early stage before they spread to lymph nodes.

Flexible endoscopy has been used to inspect and treat disorders of the gastrointestinal (GI) tract without the need for creating an opening on the patient's body. The endoscope is introduced via the mouth or anus into the upper or lower GI tracts respectively. A miniature camera at the distal end captures images of the GI wall that help the clinician in their diagnosis of the GI diseases. Simple surgical procedures (like polypectomy and biopsy) can be performed by introducing a flexible tool via a working channel to reach the site of interest at the distal end. The types of procedures that can be performed in this manner are limited by the lack of manoeuvrability of the tool. More technically demanding surgical procedures like hemostasis for arterial bleeding, suturing to mend a perforation, fundoplication for gastrooesophageal reflux cannot be effectively achieved with the conventional flexible endoscopy. These procedures are often presently being performed under open or laparoscopic surgeries.

Endoscopic submucosal dissection (ESD) has been used in treating lesions within the gastrointestinal tract. ESD is a surgery wherein a lesion part in the stomach, the intestine, and so on is dissected as a whole under an endoscopic observation. Generally, during an ESD surgery, the margins of the lesion are marked by electrocautery, and submucosal injection is used to lift the lesion; a circumferential incision into the submucosa is performed around the lesion with specialized endoscopic electrocautery knives; and the lesion is dissected from underlying deep layers of GI tract wall with the electrocautery knife and removed. Although ESD effectively removes early gastric and colorectal cancers, ESD is a technically demanding procedure associated with a higher risk of complications. For example, current flexible endoscopes may have a single instrument channel. Endoscopists can only operate with a single accessory at a time; it is difficult to maintain the tip of a flexible endoscope in a stable position inside a hollow viscus; imaging device is coupled to the instrument which may where the camera view may be blocked by the instrument's operational space. Additionally, current ESD device may have poor end-effector responsiveness, inadequate instrument capability, and usually requires significant learning curve for physicians to operate the device.

### SUMMARY OF THE INVENTION

Recognized herein is a need for an improved endoscopic submucosal dissection (ESD) system that allows for performing surgical procedures or diagnostic operations with improved patient outcomes and procedural efficiency. The present disclosure provides a modular robotic system and a robotic platform for endoscopic submucosal dissection (ESD). In particular, a modular robotic platform of the present disclosure may allow a physician to perform an endoscopic submucosal dissection (ESD) in the GI tract. The robotic platform may be configured to accommodate and manipulate the modular robotic system including a primary flexible articulatable device (e.g., primary sheath) with multiple lumens which house a variety of flexible articulatable surgical instruments. The modular robotic system may incorporate a direct visualization component, along with localization sensors for tracking position and shape of various components. This robotic platform may provide various user interfaces for controlling the ESD device. For example, the user interface may be a handheld joystick interface or a master input interface. The user interface may also provide various modalities of visualization to the user, such as real-time 2D or stereo viewers. This modular robotic endoscopic platform may allow a physician to reach a lesion in the GI tract and resect the lesion by utilizing the multiple degrees of freedom (DOF) of the flexible instruments, along with the enhanced stability and control provided to the flexible instruments by the robotic system.

In some embodiments, the primary articulatable flexible device (e.g., Gastro sheath) may comprise a plurality of lumens for a plurality of independent flexible devices or instruments. In some cases, such independent instruments can be individually deployable and articulatable. For example, the flexible instruments may each have an articulating section (e.g., wrists or bending section) allowing for additional degrees of freedom for manipulating the instruments. The articulating section may be located at the base of an end effector of the flexible instrument allowing the flexible instrument to move relative to the catheter of the primary flexible device. The term “articulating section” may be referred to as bending section which are used interchangeably throughout the specification.

The flexible instruments may have end effectors which provide surgical capabilities to the user, including but not limited to, electrosurgical hooks, scissors, forceps, needles, and graspers. The presented articulatable device and/or the modular robotic system may beneficially allow a physician to deliver surgical capabilities in an endoluminal approach, via the flexible articulatable robotic devices.

In an aspect, the present disclosure provides a robotic device. The robotic device comprises: an articulatable elongate member comprising a proximal end and a distal end, and the distal end is steerable via a first driving mechanism; an articulatable imaging instrument removably coupled to the articulatable elongate member via a first lumen of the articulatable elongate member, and the articulatable imaging instrument comprises a camera located at a distal portion of the articulatable imaging instrument; and a first articulatable instrument removably coupled to the articulatable elongate member via a second lumen, and an operation of the first articulatable instrument is captured by the camera of the articulatable imaging instrument.

In some embodiments, the robotic device further comprises a second articulatable instrument removably coupled to the articulatable elongate member via a third lumen, wherein the first articulatable instrument, the second articulatable instrument and the camera are positioned to a triangulation configuration. In some embodiments, the articulatable elongate member comprises a bending section. For instance, the bending section is articulated by one or more pull wires.

In some embodiments, the articulatable imaging instrument comprises a bending section. For instance, the bending section is articulated by one or more pull wires.

In some embodiments, the articulatable imaging instrument comprises an illuminating device located at the distal portion of the articulatable imaging instrument. In some embodiments, the articulatable imaging instrument comprises one or more nozzles for clearing a camera view. In some embodiments, the camera is controlled to roll about a longitudinal axis of the articulatable elongate member or a longitudinal axis of the articulatable imaging instrument. In some embodiments, the camera is controlled to have an articulation movement relative to the articulatable elongate member.

In some embodiments, the articulatable imaging instrument and the first articulatable instrument are withdrawn into the first lumen and the second lumen when the robotic device is in a first mode. In some cases, the articulatable imaging instrument and the first articulatable instrument are extended out of the distal end of the articulatable elongate member when the robotic device is in a second mode.

In some embodiments, the articulatable imaging instrument is steerable via the first driving mechanism. In some embodiments, the first driving mechanism is mounted to a first robotic support system. In some cases, the first articulatable instrument is articulated via a second driving mechanism. In some instances, the second driving mechanism is mounted to a second robotic support system. For example, the first robotic support system and the second robotic support system are operatively coupled to control the robotic device. In some embodiments, the proximal end of the articulatable elongate member is removably coupled to the first driving mechanism.

In another aspect, the present disclosure provides a method for operating the modular robotic device. The method comprises: providing an articulatable elongate member such as a primary sheath comprising a first lumen and a second lumen; coupling an articulatable imaging instrument to the articulatable elongate member via the first lumen; coupling a first articulatable instrument to the articulatable elongate member via the second lumen; and capturing an operation of the first articulatable instrument by the camera of the articulatable imaging instrument. In some cases, the primary sheath comprises a third lumen to accept a second articulatable instrument. The camera is located at a distal portion of the articulatable imaging instrument and is individually controlled to have at least articulation movement relative to the primary sheath.

In some embodiments, the method further comprises coupling a second articulatable instrument to the articulatable elongate member via a third lumen. In some embodiments, the camera is controlled to have articulating movement relative to the articulatable elongate member. In some embodiments, the articulatable elongate member is steered via a first driving mechanism. In some cases, the first articulatable instrument is actuated via a second driving mechanism. In some instances, the first driving mechanism and the second driving mechanism are operatively coupled. In some embodiments, the articulatable imaging instrument is articulated and manipulated via the first driving mechanism.

It should be noted that the provided modular robotic systems and various components of the robotic platform can be used in various minimally invasive surgical procedures, therapeutic or diagnostic procedures that involve various types of tissue including heart, bladder and lung tissue, and in other anatomical regions of a patient's body such as a digestive system, including but not limited to the esophagus, liver, stomach, colon, urinary tract, or a respiratory system, including but not limited to the bronchus, the lung, and various others.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a modular robotic system, in accordance with some embodiments of the present disclosure.

FIG. 2 shows an example of a modular robotic system in a deployed mode, in accordance with some embodiments of the present disclosure

FIG. 3 shows examples of flexible articulatable instruments, in accordance with embodiments of the present disclosure.

FIG. 4 shows examples of a robotic platform.

FIG. 5 shows an example of a robotic platform with assembled control modules.

FIG. 6 shows an example of a primary articulatable flexible device supported by a robotic support system.

FIG. 7 shows an example of an instrument driving mechanism providing mechanical interface to a handle portion.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

While exemplary embodiments will be primarily directed at a device or system for endoscopic submucosal dissection (ESD), one of skill in the art will appreciate that this is not intended to be limiting, and the devices described herein may be used for other therapeutic or diagnostic procedures and in various anatomical regions of a patient's body such as a digestive system, including but not limited to the esophagus, liver, stomach, colon, urinary tract, or a respiratory system, including but not limited to the bronchus, the lung, and various others.

The embodiments disclosed herein can be combined in one or more of many ways to provide improved diagnosis and therapy to a patient. The disclosed embodiments can be combined with existing methods and apparatus to provide improved treatment, such as combination with known methods of pulmonary diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

Although the robotic system, definition of diagnosis or surgical procedures as described herein are presented in the context of diagnosis or surgery for gastrointestinal (GI) tract, the methods and apparatus as described herein can be used to treat any tissue of the body and any organ and vessel of the body such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein a processor encompasses one or more processors, for example a single processor, or a plurality of processors of a distributed processing system for example. A controller or processor as described herein generally comprises a tangible medium to store instructions to implement steps of a process, and the processor may comprise one or more of a central processing unit, programmable array logic, gate array logic, or a field programmable gate array, for example. In some cases, the one or more processors may be a programmable processor (e.g., a central processing unit (CPU) or a microcontroller), digital signal processors (DSPs), a field programmable gate array (FPGA) and/or one or more Advanced RISC Machine (ARM) processors. In some cases, the one or more processors may be operatively coupled to a non-transitory computer readable medium. The non-transitory computer readable medium can store logic, code, and/or program instructions executable by the one or more processors unit for performing one or more steps. The non-transitory computer readable medium can include one or more memory units (e.g., removable media or external storage such as an SD card or random access memory (RAM)). One or more methods or operations disclosed herein can be implemented in hardware components or combinations of hardware and software such as, for example, ASICs, special purpose computers, or general purpose computers.

As used herein, the terms distal and proximal may generally refer to locations referenced from the apparatus, and can be opposite of anatomical references. For example, a distal location of a primary sheath or catheter may correspond to a proximal location of an elongate member of the patient, and a proximal location of the primary sheath or catheter may correspond to a distal location of the elongate member of the patient.

In an aspect of the present disclosure, a modular robotic system is provided. The modular robotic system may comprise a primary articulatable flexible device (e.g., Gastro sheath) with removably coupled real-time endoscopic vision and multiple instruments for performing intricate and precise surgical operations. The modular robotic system may provide real-time endoscopic view, thus providing the advantage to the endoscopist for performing intricate and difficult surgical procedures using natural orifices to access the internal organs. In particular, the GI tract may eliminate any scars on the patient.

In some embodiments, the primary articulatable flexible device (e.g., Gastro sheath) may comprise a plurality of lumens for housing a plurality of independent flexible devices or instruments. In some cases, such independent instruments can be individually deployable and articulatable. For example, the flexible instruments may each have an articulating section (e.g., wrists or bending section) allowing for additional degrees of freedom (DOFs) for manipulating the instruments. The articulating section may be located at the base of an end effector of the flexible instrument allowing the flexible instrument to move relative to the catheter of the primary flexible device. The flexible instruments may have end effectors which provide surgical capabilities to the user, including but not limited to, electrosurgical hooks, scissors, forceps, needles, and graspers. The presented articulatable device and/or the modular robotic system may beneficially allow a physician to deliver surgical capabilities in an endoluminal approach, via the flexible articulatable robotic devices.

The modular robotic system or the primary articulatable flexible device as described herein, includes an elongate portion or elongate member such as a catheter. The terms “elongate member,” “catheter,” “sheath” are used interchangeably throughout the specification unless contexts suggest otherwise. The elongate member can be placed directly into the body lumen or a body cavity. A handle portion or proximal portion of the modular robotic system may be placed outside of the body cavity. The sheath or catheter may comprise an articulating section and control mechanism for steering and articulatable the device.

The modular robotic system may be coupled to a support apparatus such as a robotic manipulator (e.g., robotic arm) for driving, supporting, positioning or controlling the movements and/or operation of the modular robotic system. Alternatively or in addition to, the modular robotic system can be controlled via a hand-held device or other control devices that may or may not include a robotic system. In some embodiments, the robotic system may further include peripheral devices and subsystems such as imaging systems that would assist and/or facilitate the navigation of the elongate member to the target site in the body of a subject.

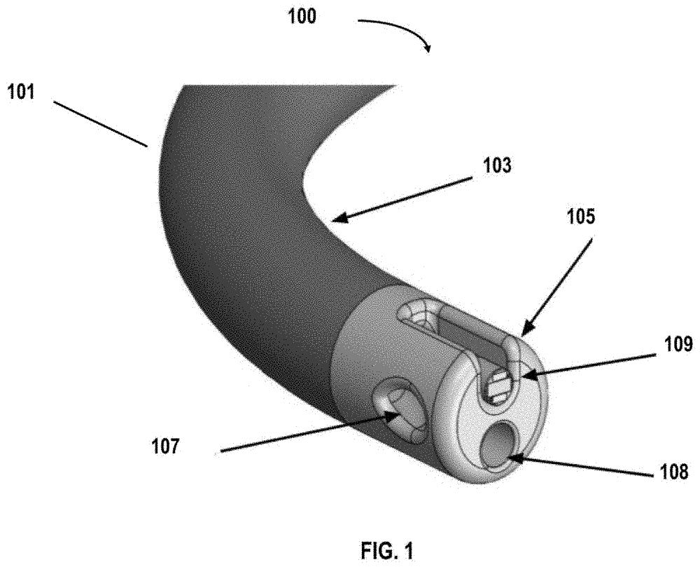

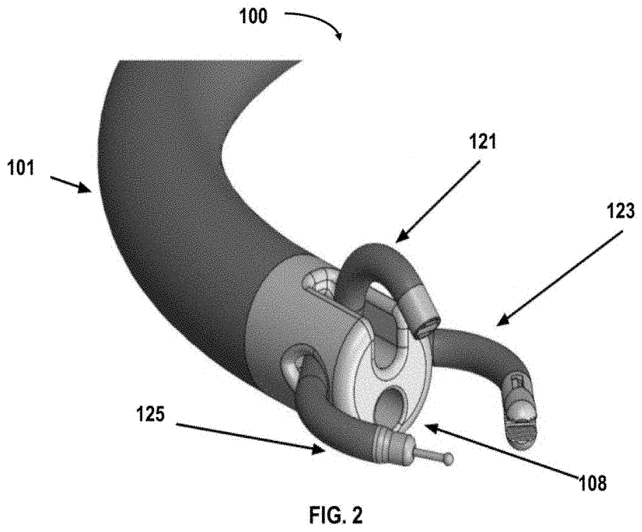

FIG. 1 and FIG. 2 show an example of a modular robotic system 100, in accordance with some embodiments of the present disclosure. As shown in the figures, the modular robotic system 100 may comprise a primary sheath or primary articulatable flexible device 101, a plurality of flexible and steerable instruments 123, 125 and an endoscopic instrument 121. In some cases, the endoscopic instrument 121 may be integrated to the primary articulatable flexible device. For example, the endoscopic instrument 121 may be integral to the primary articulatable flexible device. In alternative cases, the endoscopic instrument 121 may be removably coupled to the primary articulatable flexible device. The plurality of flexible and steerable instruments 123, 125 and the endoscopic device 121 may be coupled to the modular robotic system as an assembly with at least a portion of the instruments or endoscopic instrument movable relative to the primary sheath. In some cases, the plurality of flexible and steerable instruments 123, 125 may be removably coupled to the primary sheath.

As shown in FIG. 1, the primary sheath 101 may be a probing portion that is brought into proximity to the tissue and/or area that is to be examined. The primary sheath 101 may be steerable and robotically controlled. The robotic control module and user interface for controlling the primary sheath are described later herein.

The primary sheath may be composed of suitable materials for desired flexibility or bending stiffness. In some cases, the materials of the sheath may be selected such that it may maintain structural support to the internal structures (e.g., working channel) as well as being substantially flexible (e.g., able to bend in various directions and orientations). For example, the catheter can be made of any suitable material such as Provista Copolymer, vinyl (such as polyvinyl chloride), Nylon (such as vestamid, grillamid), pellethane, polyethylene, polypropylene, polycarbonate, polyester, silicon elastomer, acetate and so forth. In some cases, the materials may be polymer material, bio-compatible polymer material and the catheter may be sufficiently flexible to be advancing through a path with a small curvature without causing pain to a patient.

The primary sheath may comprise a shaft, an articulation section 103 and a steerable distal portion 105, where articulation section (bending section) 103 is connecting the steerable distal portion to the shaft. For example, the bending section may be connected to the distal tip portion at a first end, and connected to a shaft portion at a second end, where the bending section is articulated by one or more pull wires. In some cases, the bending section may be fabricated separately as a modular component and assembled to the shaft. In some cases, the bending section may further incorporate minimalist features thereby reducing cost and increasing reliability. For example, the bending section may incorporate a cut pattern that beneficially allows for a greater degree of tube deflection to achieve a desired tip displacement relative to the shaft. In some cases, the bending section may be composed of stainless steel ribbon. The bending section may be formed of other suitable structures or materials to achieve pre-determined bending stiffness while maintaining desired axial and torsional stiffness with low articulation force. For example, the bending section may comprise braid structures for torsional stability.

The distal portion of the primary sheath may be steered by control elements such as one or more pull wires, gears, pulleys or other driving mechanism. The distal portion 105 of the primary sheath may be made of any suitable material such as co-polymers, polymers, metals or alloys and it can be steered by the pull wires. In some cases, the distal tip 105 may be a rigid component that allow for positioning sensors such as electromagnetic (EM) sensors being embedded at the distal tip.

In some cases, the distal portion 105 may be configured to be articulated/bent in two or more degrees of freedom to provide a desired camera view together with an articulatable endoscopic instrument or control the direction of the endoscope. In some embodiments, the proximal end or portion of one or more pull wires may be operatively coupled to various mechanisms (e.g., gears, pulleys, etc.) in a handle/proximal portion of the robotic assembly. In some cases, the pull wires may be anchored at the distal tip of the primary sheath, running through the bending section, and entering the handle where they are coupled to a driving component (e.g., pulley).

The pull wire may be a metallic wire, cable or thread, or it may be a polymeric wire, cable or thread. The pull wire can also be made of natural or organic materials or fibers. The pull wire can be any type of suitable wire, cable or thread capable of supporting various kinds of loads without deformation, significant deformation, or breakage. The distal end or distal portion of the one or more pull wires may be anchored or integrated to the distal portion 105 of the primary sheath, such that operation of the pull wires by the control unit may apply force or tension to the distal portion 105 which may steer or articulate (e.g., up, down, pitch, yaw, or any direction in-between) at least the distal portion (e.g., flexible section) 105 of the primary sheath.

The modular robotic system 100 may be configured to have at least an endoscope mode (e.g., colonoscope mode) such as shown in FIG. 1 and a deployed mode such as shown in FIG. 2. In the endoscope mode, the plurality of flexible instruments 123, 125 and the articulatable endoscopic instrument 121 may be partially or completely withdrawn into the primary sheath 101 such as when the primary sheath is being advanced to a target site inside a patient (e.g., colon intubation phase) or retracted from the target site (e.g., colonoscope retraction). In some cases, when the modular robotic system is in the endoscope mode such as when it is navigating to the target site, the location and orientation of the distal end of the endoscope may be tracked by the EM sensor and the camera. The camera may be located at the articulatable endoscope instrument 121 which is withdrawn inside the primary sheath. The EM sensor may be embedded in the distal tip 105 and/or located at the articulatable endoscope instrument 121.

Once the primary sheath 100 is advanced to a location near the target site, the flexible and steerable arms of the flexible instruments 123, 125 and the articulatable endoscope instrument 121 may be advanced/extended out of the ports on the primary sheath and further steered or maneuvered into position to perform various diagnostic or therapeutic operations. Each of the steerable arm may comprise a flexible shaft, a bending section allowing for articulation of the tip of the flexible instruments 123, 125 or articulatable endoscope instrument 121. The above description about the bending section and pull wires are applicable to the flexible instruments 123, 125 or articulatable endoscope instrument 121.

The primary sheath 101 may comprise a plurality of lumens 107, 108, 109. As described above, the appropriate surgical instruments may be advanced through each lumen of the instrument assemblies to execute the various diagnostic or therapeutic operations. In some cases, a first lumen 105 may accommodate an independent articulatable endoscope instrument 121. The articulatable endoscope instrument 121 may allow for the field of view to be maneuvered or controlled relative to the primary sheath 101 or the distal tip 105. This may beneficially provide a user with improved flexibility and capability to optimize their viewable workspace or field of view without compromising the position or stability of the instruments, primary sheath and anatomy.

Two of the lumens 107 may accommodate a flexible electrosurgical instrument 123, 125 such as forceps, graspers, surgical clip appliers, injection needles, or scissors and the like. The flexible instruments may be controlled to insert, retract, and rotate relative to the primary sheath. Such additional degrees of freedom of the instruments may beneficially minimize the risk of compromising the anatomical fixation of the primary sheath during the instrument interaction with a GI lesion.

The primary sheath may have any suitable dimension so that the lumens may house the plurality of flexible instruments. For example, the outer diameter of the distal tip may be around 20 millimeters (mm), and the diameter of one or more of the lumens may be around 6 mm. However, it should be noted that based on different applications, the outer diameter can be in any range smaller than 20 mm or greater than 20 mm, and the diameter of the lumens or working channel can be in any range according to the tool dimensional or specific application.

In some embodiments, the primary sheath may comprise an additional working channel/tool port 108 to accommodate additional controllable instrument assembly. As an example, a working channel 108 may have a dimension such as diameter of around 2 mm or 6 mm to be compatible with standard tools.

The primary sheath may comprise fewer or more lumens. In some embodiments, the primary sheath may comprise two lumens for the flexible instruments 123, 125 whereas the imaging device (e.g., camera) may be embedded into the distal portion 105 of the primary sheath. In some cases, the imaging device may be embedded into the distal portion of the primary sheath. In some cases, the imaging device may be coupled to the distal portion 105 of the primary sheath whereas the viewing angle can be tilted or rotated relative to the distal portion. In some cases, one or more electronic components can be integrated to the distal tip of the primary sheath. For example, a camera and/or a positional sensor (e.g., electromagnetic sensor) can be embedded into the distal tip 105.

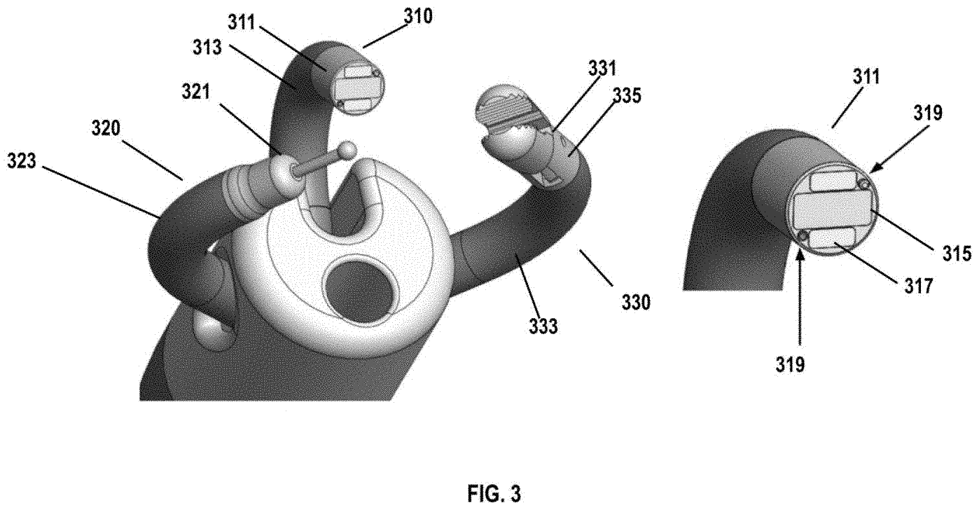

FIG. 3 shows examples of flexible articulatable instruments, in accordance with some embodiments of the present disclosure. In some embodiments, the plurality of flexible articulatable instruments may include at least an articulatable endoscope instrument 310 and one or more surgical instruments each has a robotic arm 323, 333, and end effectors or instrument tools 321, 331, which may be extended from the ports on the primary flexible instrument body.

The articulatable endoscope instrument 310 may comprise a steerable and articulatable arm 313 and a distal tip 311 where one or more electronics are located. The imaging device or camera is controlled to have an articulation movement relative to the primary sheath. The articulatable arm may be a robotic arm that can be robotically controlled. The one or more electronics may include at least an imaging device 315, and an illuminating device 317.

In some cases, the articulatable imaging instrument comprises one or more nozzles for clearing a camera view. For instance, the distal tip may further comprise one or more irrigation ports such as a forward irrigation nozzle 319 and a window cleaning nozzle for providing a clear camera view. For example, an irrigation and aspiration system may connect to the working channel for the articulatable endoscope instrument through a connector or a lure. The irrigation system can inject fluids such as saline and the aspiration system may aspire mucus or saline or other material out of the airways.

The imaging device 315 may be a camera for direct vision. The imaging device may be located at the distal tip of the articulatable endoscope instrument 310. In some embodiments, the imaging device may be a video camera. The imaging device may comprise optical elements and image sensor for capturing image data. The image sensors may be configured to generate image data in response to wavelengths of light. A variety of image sensors may be employed for capturing image data such as complementary metal oxide semiconductor (CMOS) or charge-coupled device (CCD). The imaging device may be a low-cost camera. In some cases, the image sensor may be provided on a circuit board. The circuit board may be an imaging printed circuit board (PCB). The PCB may comprise a plurality of electronic elements for processing the image signal. For instance, the circuit for a CCD sensor may comprise A/D converters and amplifiers to amplify and convert the analog signal provided by the CCD sensor. Optionally, the image sensor may be integrated with amplifiers and converters to convert analog signal to digital signal such that a circuit board may not be required. In some cases, the output of the image sensor or the circuit board may be image data (digital signals) can be further processed by a camera circuit or processors of the camera. In some cases, the image sensor may comprise an array of optical sensors. As described elsewhere herein, the imaging device may be located at the distal tip of the independent endoscope instrument 310 or is embedded into the distal tip of the primary sheath.

The illumination device 317 may comprise one or more light sources positioned at the distal tip of the articulatable endoscope instrument 310. The light source may be a light-emitting diode (LED), an organic LED (OLED), a quantum dot, or any other suitable light source. In some cases, the light source may be miniaturized LED for a compact design or Dual Tone Flash LED Lighting.

The flexible endoscope instrument 310 may be independently controlled to articulate, roll, insert, retract and the like relative to the primary sheath. For example, the flexible endoscope instrument assembly may be controlled to roll and insert relative to the primary sheath, while the distal segment exits the distal portion of the primary sheath, the distal portion 311 of the flexible endoscope instrument may be directed and orientated by controlling the articulation of the arm 313 and/or a rotational movement of the flexible endoscope instrument. The rotational movement can be achieved by rotating an elongate body of the flexible endoscope instrument relative to the primary sheath and/or the distal base at the distal tip 311. For example, the camera may have a roll movement with respect to the primary sheath by rotating the flexible endoscope instrument assembly about the longitudinal axis of the primary sheath. Alternatively or additionally, the flexible endoscope instrument assembly may comprise a ratable wrist allowing the camera to roll about the longitudinal axis of the flexible endoscope instrument assembly. Alternatively, the camera view may be rotated via imaging processing (e.g., to be aligned to gravity direction). The articulation may be controlled in a similar manner to the primary articulatable sheath. For example, individual pull wires and other control elements may be provided for controlling the movement of the flexible endoscope instrument 310. As described above, the articulatable arm 313 may comprise a bending section that can be articulated in a manner similar to the primary sheath.

As illustrated in the example, the camera may be positioned and oriented with improved flexibility and working space such that real-time view of an operation scene can be provided at any varied angles and vantage points. By providing the camera at the tip of the articulatable arm that is individually controlled with respect to the primary sheath, the view of operation scene can be provided without affecting the operation of the robotic manipulator of the other instruments 320, 330. This may beneficially allow for an operation environment being clearly viewed by the surgeon from a user-selected angle. For example, user may freely adjust the vantage point, location, field of view of the camera without affecting the operation of the instruments.

The two flexible instruments 320, 330 may each comprise a robotic arm 323, 333 including a proximal segment and a distal segment. In some cases, the robotic arms include a proximal base (not shown), a distal base 335, and a distal tip 331. The distal tip 331 may carry any suitable tools such as grasper or other electrosurgical instruments as described elsewhere herein. In some cases, the tools may be suitable for performing Endoscopic Submucosal Dissection (ESD). The proximal base, distal base, and distal tip may be controlled by control elements of the corresponding robotic arm. For instance, the control elements may include pull wires and other control elements as described elsewhere herein.

The two flexible instruments 320, 330 may fulfill the flexibility, dexterity and triangulation requirement for endoluminal operations. In a preferred configuration, the two instrument ports may be located on opposite sides of the port for the endoscope instruments. This configuration may allow for surgical triangulation with the distal portions of the instrument assemblies.

The flexibility offered by the articulatable arm of the endoscope instrument and the two flexible instruments may beneficially allow for the instruments to perform triangulation and converge on the area of interest. For instance, by orienting the camera on top and an instrument at each lower point of substantially a triangle creating a converging triad, the sight of the instruments and operating efficiency are maximized. For instance, the additional degrees of freedoms may allow the flexible instruments 320 and 330 to have triangulation by making the arms spread out or divert away from the base as they exit the distal portion of the primary sheath. The distal portions may then be steered back toward each other and utilized to apply capturing and/or compressive loads to a subject tissue structure, and the like, with the field of view of the image capture device preferably capturing such activity from any desired location relative to the instruments 320 330. In this manner, the arms of the robotic manipulator do not block the endoscopic view so that the operation environment could be clearly viewed by the surgeon and the camera can be individually maneuvered such that the real-time imaging can be captured from any desired vantage point relative to the operation scene without comprising the operation of the instruments.

In an aspect, the present disclosure provides a method for operating the modular robotic device. The method comprises: providing an articulatable elongate member such as a primary sheath comprising a first lumen and a second lumen; coupling an articulatable imaging instrument to the articulatable elongate member via the first lumen; coupling a first articulatable instrument to the articulatable elongate member via the second lumen; and capturing an operation of the first articulatable instrument by the camera of the articulatable imaging instrument. In some cases, the primary sheath comprises a third lumen to accept a second articulatable instrument. The camera is located at a distal portion of the articulatable imaging instrument and is individually controlled to have at least articulation movement relative to the primary sheath.

### Robotic Platform

In some embodiments, a robotic platform may be provided allowing a physician to perform an Endoscopic Submucosal Dissection (ESD) in the GI tract. The platform may comprise a modular robotic system as described above housing a variety of flexible articulatable surgical instruments, and a support apparatus such as a robotic manipulator (e.g., robotic arm) to drive, support, position or control the movements and/or operation of the modular robotic system. the robotic platform may further include peripheral devices and subsystems such as imaging systems that would assist and/or facilitate the navigation of the elongate member to the target site in the body of a subject.

In some cases, the modular robotic system may also implement a positional sensing system such as electromagnetic (EM) sensor, fiber optic sensors, and/or other sensors to register and display a medical implement together with preoperatively recorded surgical images thereby locating a distal portion of the endoscope with respect to a patient body or global reference frame. The position sensor may be a component of an EM sensor system including one or more conductive coils that may be subjected to an externally generated electromagnetic field. Each coil of EM sensor system used to implement positional sensor system then produces an induced electrical signal having characteristics that depend on the position and orientation of the coil relative to the externally generated electromagnetic field. In some cases, an EM sensor system used to implement the positional sensing system may be configured and positioned to measure at least three degrees of freedom e.g., three position coordinates X, Y, Z. Alternatively or in addition to, the EM sensor system may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. The position sensor may be embedded into the distal tip of the primary articulatable flexible device, the integrated flexible and steerable instruments, and/or the integrated endoscopic instrument as described above. The flexible and steerable instruments and/or the endoscopic instrument may be integrated in a removable fashion or integral to the primary articulatable flexible device.

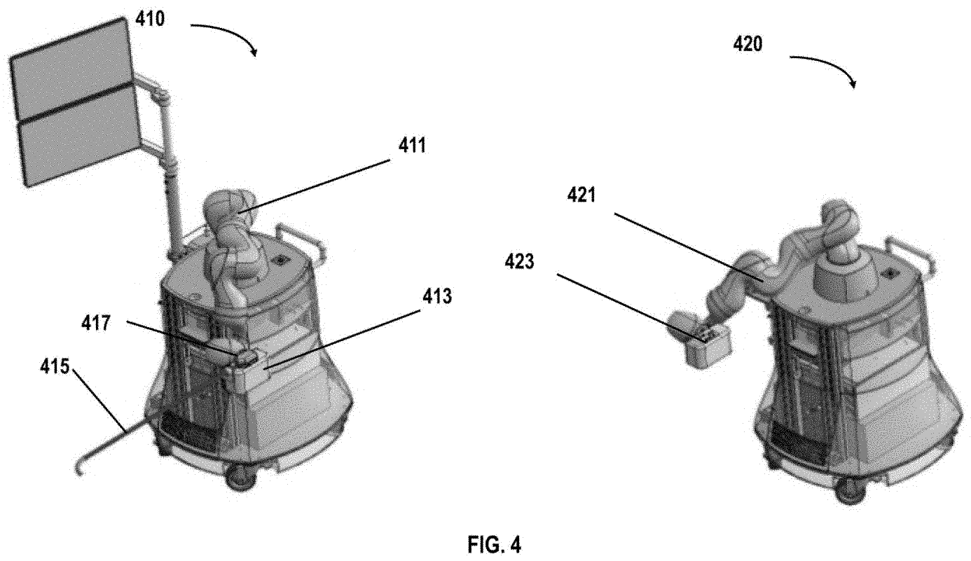

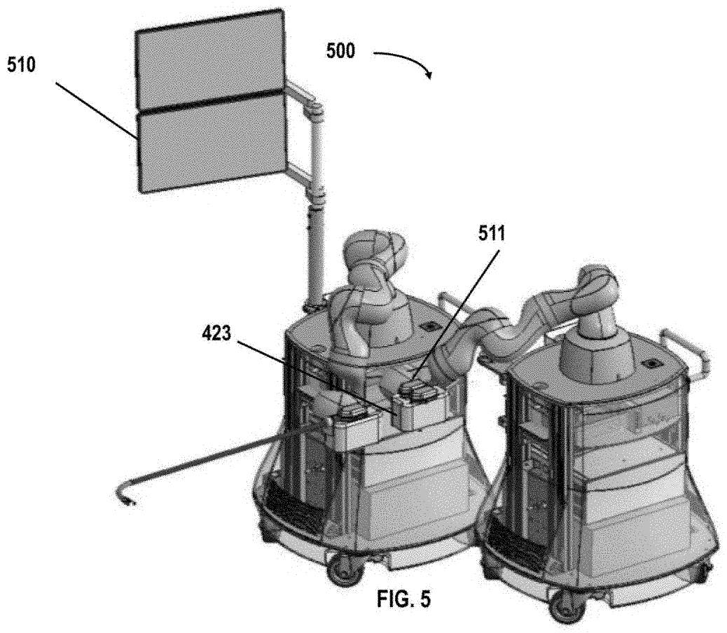

FIG. 4 and FIG. 5 show examples of a robotic platform 430. In some embodiments, the robotic platform may include a first control modules 410 for controlling operations of the primary sheath for endoscopic functionalities (e.g., colon intubation, retraction, etc.) and a second control module 420 for controlling operations of the instruments (e.g., ESD operations). The first control module and the second control module may be removably coupled to form a control system 500 of the robotic platform as shown in FIG. 5.

In some embodiments, each control module 410, 420 may include or be integrated with a robotic support system including a robotic arm 411, 421, instrument driving mechanism 413, 423, robotic control unit, and one or more peripherical equipment's such as irrigation and aspiration system. The robotic arm of the first control module 410 may initiate the positioning of the modular robotic system or other robotic instrument. The robotic arm 411 may automatically position the modular robotic assembly 415 to an initial position (e.g., access point) to access the target tissue. In some embodiments, the robot arm can be passively moved by an operator. In such case, an operator can push the arm in any position and the arm compliantly moves. The robot can also be controlled in a compliant mode to improve human robot interaction. For example, the compliant motion control of the robot art may employ a collision avoidance strategy and the position-force control may be designed to save unnecessary energy consumption while reducing impact of possible collisions. The arm may have redundant degrees of freedom allowing for its elbow to be algorithmically, or passively, moved into configurations that are convenient for an operator.

In some embodiments, the instrument driving mechanism 413 may be mounted to the robotic arm 411. The modular robotic system 415 can be releasably coupled to the instrument driving mechanism 413. The instrument driving mechanism may be mounted to the arm of the robotic support system or to any actuated support system. The instrument driving mechanism may provide mechanical and electrical interface to the modular robotic system 415. The mechanical interface may allow the modular robotic system 415 to be releasably coupled to the instrument driving mechanism. For instance, the handle portion of the modular robotic system 415 can be attached to the instrument driving mechanism via quick install/release means, such as magnets and spring-loaded levels. In some cases, the modular robotic system 415 may be coupled or released from the instrument driving mechanism manually without using a tool. The instrument driving mechanism 413 may be used to drive the primary sheath in two or more degrees of freedom (e.g., articulation) and other movement as described elsewhere herein.

The modular robotic system 415 can be releasably coupled to the instrument driving mechanism 413 via a handle portion 417. For example, the pull wires of the primary sheath may run through the bending section, the sheath and enter the handle where they are coupled to a driving component (e.g., pulley). This handle pulley may interact with an output shaft in the instrument driving mechanism.

In some case, the handle portion 417 may be housing or comprise components configured to process image data, provide power, or establish communication with other external devices. In some cases, the communication may be wireless communication. For example, the wireless communications may include Wi-Fi, radio communications, Bluetooth, IR communications, or other types of direct communications. Such wireless communication capability may allow the modular robotic system function in a plug-and-play fashion and can be conveniently disposed after single use. In some cases, the handle portion may comprise circuitry elements such as power sources for powering the electronics (e.g. camera and LED light source) of the modular robotic system.

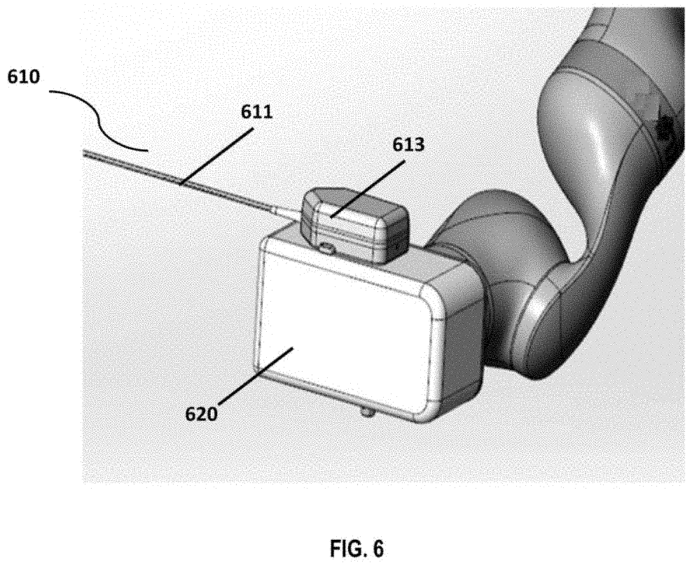

FIG. 6 shows an example of a primary articulatable flexible device 610 supported by a robotic support system. The primary articulatable flexible device and the robotic support system can be the same as those described above. For example, the primary articulatable flexible device may comprise an elongate member 611 and a handle portion 613. In some embodiments, the primary articulatable flexible device 610 may also comprise an imaging device and/or positional sensor(s) integrated to the distal portion of the elongate member. Alternatively, the primary articulatable flexible device 610 may be coupled to an endoscopic instrument to provide endoscopic functions. The elongate member 611 may comprise a flexible shaft, a bending section connecting the shaft to a steerable tip, and a plurality of lumens for housing a plurality of removable flexible devices or instruments. The elongate member 611 can be the same as the primary sheath as described above.

The handle portion 613 may be in electrical communication with one or more electronic components coupled to the elongate member 611. For example, when an imaging device, illuminating device and/or EM sensor are integrated to the elongate member 611, image/video data and/or sensor data may be transmitted to one or more processors in the handle portion. In some case, the handle portion may be housing or comprise components configured to process image data, provide power, or establish communication with other external devices. In some cases, the communication may be wireless communication. For example, the wireless communications may include Wi-Fi, radio communications, Bluetooth, IR communications, or other types of direct communications. Such wireless communication capability may allow the modular robotic system or primary articulatable flexible device function in a plug-and-play fashion and can be conveniently disposed after single use. In some cases, the handle portion may comprise circuitry elements such as power sources for powering the electronics (e.g. camera and LED light source) disposed within the modular robotic device or the primary sheath.

In some cases, the handle portion 613 may in electrical communication with one or more electronic components that are not integrated to the primary sheath. For instance, when the imaging device, EM sensor and other electronic components are located at the removable endoscopic instrument, a proximal end of the endoscopic instrument may be in electrical communication with the handle portion 613 or may be connected to the handle portion 613.

In some cases, the handle portion may be in electrical communication with an instrument driving mechanism (e.g., instrument driving mechanism 620) via an electrical interface (e.g., printed circuit board) so that image/video data and/or sensor data can be received by the communication module of the instrument driving mechanism and may be transmitted to other external devices/systems. In some cases, the electrical interface may establish electrical communication without cables or wires. For example, the interface may comprise pins soldered onto an electronics board such as a printed circuit board (PCB). For instance, receptacle connector (e.g., the female connector) is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data and/or receiving control signals.

In some embodiments, the flexible elongate member 611 may comprise a shaft, steerable tip, a articulating section and multiple lumens to receive the plurality of flexible and steerable instruments and/or the endoscopic instrument as described above. The primary articulatable flexible device 610 can be the same as the primary sheath or primary articulatable flexible device as described in FIG. 1 and FIG. 2. In some cases, the primary articulatable flexible device 610 may be a single-use device. In some cases, only the catheter may be disposable. In some cases, at least a portion of the catheter may be disposable. In some cases, the entire primary articulatable flexible device 610 may be released from the instrument driving mechanism and can be disposed of. In some cases, the primary articulatable flexible device may contain varying levels of stiffness along its shaft, as to improve functional operation.

The primary articulatable flexible device 610 can be releasably coupled to an instrument driving mechanism 620. The instrument driving mechanism 620 may be mounted to the arm of the robotic support system or to any actuated support system as described elsewhere herein. The instrument driving mechanism may provide mechanical and electrical interface to the primary articulatable flexible device 620. The mechanical interface may allow the primary articulatable flexible device 620 to be releasably coupled to the instrument driving mechanism. For instance, the handle portion of the primary articulatable flexible device 620 can be attached to the instrument driving mechanism via quick install/release means, such as magnets and spring-loaded levels. In some cases, the primary articulatable flexible device 620 may be coupled or released from the instrument driving mechanism manually without using a tool. It should be noted that any description about the handle portion or the instrument driving mechanism about the primary articulatable flexible device is applicable to the handle portion or the instrument driving mechanism for the plurality of articulable instruments.

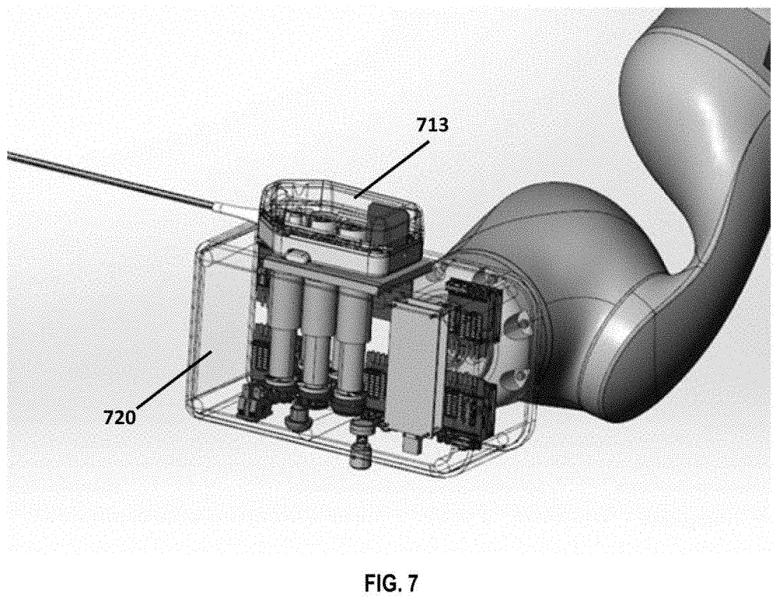

FIG. 7 shows an example of an instrument driving mechanism 720 providing mechanical interface to the handle portion 713 of the primary articulatable flexible device or the modular robotic system. As shown in the example, the instrument driving mechanism 720 may comprise a set of motors that are actuated to rotationally drive a set of pull wires of the catheter. The handle portion 713 may be mounted onto the instrument drive mechanism so that its pulley assemblies are driven by the set of motors. The number of pulleys may vary based on the pull wire configurations. In some cases, one, two, three, four, or more pull wires may be utilized for articulating the catheter.

The handle portion may be designed allowing the primary articulatable flexible device to be disposable at reduced cost. For instance, classic manual and robotic endoscopes may have a cable in the proximal end of the endoscope handle. The cable often includes illumination fibers, camera video cable, and other sensors fibers or cables such as electromagnetic (EM) sensors, or shape sensing fibers. Such complex cable can be expensive adding to the cost of the endoscope. The provided modular robotic system or primary articulatable flexible device may have an optimized design such that simplified structures and components can be employed while preserving the mechanical and electrical functionalities. In some cases, the handle portion e may employ a cable-free design while providing a mechanical/electrical interface to the catheter.

The irrigation and aspiration systems may reside on a robotic arm base cart or any other part of the system. The irrigation and aspiration system may connect to the working channel for the articulatable endoscope instrument through a connector or a lure. The irrigation system can inject fluids such as saline and the aspiration system may aspire mucus or saline or other material out of the airways. As described above, the irrigation and aspiration system may be used for camera visualization.

In some embodiments, the first control module 410 and the second control module 420 may collectively control the modular robotic system 415. In some embodiments, the instrument driving mechanism and the robotic control unit of the first control module 410 may be configured to control and manipulate the primary sheath and the integrated endoscopic instrument (e.g., camera). The instrument driving mechanism 423 and robotic control unit of the second control module 420 may be used for manipulating the multiple flexible instruments such as the pair of instruments for performing the ESD. For instance, articulation, insertion, retraction and various other movement of the flexible instruments are driven by the instrument driving mechanism 423. As illustrated in FIG. 5, the driving mechanism 423 of the second control module may be coupled to the driving mechanism 413 of the first control module thereby driving the multiple instruments of the modular robotic system. For example, a proximal portion or handle 511 of the flexible instruments may be connected to the instrument driving mechanism 423 to drive the one or more pull wires of the flexible instruments. In some cases, the instrument driving mechanism 423 of the second control module and the instrument driving mechanism 413 of the first control module may be operatively coupled. For instance, the two instrument driving mechanisms may be robotically controlled to move synchronously to collectively control the modular robotic system 415.

The robotic platform 500 may comprise a user interface 510 located at the patient and robot side. The user interface may allow an operator or user to interact with the robotic system during surgical procedures. In some embodiments, the user interface 510 may be implemented on a hand-held controller. The user interface 510 may, in some cases, comprise a proprietary user input device and one or more add-on elements removably coupled to an existing user device to improve user input experience. For instance, physical trackball or roller can replace or supplement the function of at least one of the virtual graphical elements (e.g., navigational arrow displayed on touchpad) displayed on a graphical user interface (GUI) by giving it similar functionality to the graphical element which it replaces. Examples of user devices may include, but are not limited to, mobile devices, smartphones/cellphones, tablets, personal digital assistants (PDAs), laptop or notebook computers, desktop computers, media content players, and the like. In some cases, the user interface 510 may provide real-time vision and visional guidance allowing a physician to reach a lesion in the GI tract and resect the lesion by utilizing the multiple degrees of freedom (DOF) of the instrumentation, along with the enhanced stability and control provided to the instruments by the robotic system.

In some embodiments, the robotic system may include a navigation and localization subsystem configured to construct a virtual airway model based on the pre-operative image (e.g., pre-op CT image). The navigation and localization subsystem may be configured to identify an approximate segmented lesion location in the 3D rendered airway model and based on the location of the lesion, the navigation and localization subsystem may generate an optimal path to the lesions in the GI tract with a recommended approaching angle towards the lesion for performing surgical procedures (e.g., ESD). For example, a processing unit may be configured to generate an augmented layer comprising augmented information such as the location of the treatment location or the lesion. In some cases, the augmented layer may also comprise graphical marker indicating a path to this target site. The augmented layer may be a substantially transparent image layer comprising one or more graphical elements (e.g., box, arrow, etc). The augmented layer may be superposed onto the optical view of the optical images or video stream captured by the fluoroscopy (tomosynthesis) imaging system, and/or displayed on the display device. The transparency of the augmented layer allows the optical image to be viewed by a user with graphical elements overlay on top of. In some cases, both the segmented lesion images and an optimum path for navigation of the elongate member to reach the lesion may be overlaid onto the virtual airway model or pre-operative images. This may allow operators or users to visualize the approximate location of the lesion as well as a planned path of the primary sheath movement. In some cases, the segmented and reconstructed images (e.g. CT images) provided prior to the operation of the systems may be overlaid on the real time images.

At a registration step before driving the modular robotic system to the target site, the system may align the rendered virtual view of the airways to the patient airways. Image registration may consist of a single registration step or a combination of a single registration step and real-time sensory updates to registration information. Once registered, all airways may be aligned to the pre-operative rendered airways. During the modular robotic system driving towards the target site, the location of the primary sheath inside the airways may be tracked and displayed. In some cases, location of the tip of the primary sheath with respect to the airways may be tracked using positioning sensors. Other types of sensors (e.g. camera) can also be used instead of or in conjunction with the positioning sensors using sensor fusion techniques. Positioning sensors such as electromagnetic (EM) sensors may be embedded at the distal tip of the primary sheath/or the flexible endoscope instrument (e.g., next to the camera) and an EM field generator may be positioned next to the patient torso during procedure. The EM field generator may locate the EM sensor position in 3D space or may locate the EM sensor position and orientation in 5D or 6D space. This may provide a visual guide to an operator when driving the robotic system towards the target site.

During operation, the lesion location and various operations of the one or more flexible instruments may be tracked in real-time by the camera. In some embodiments, the user interface may include, for example, a user interface hand held device allowing physicians to control the robotic endoscope (e.g. colonoscope) with ease.

In some cases, the user interface, the robotic control modules, and the robotic arm may be mounted to a separate mobile cart. The mobile cart may include various elements such as rechargeable power supply in electrical communication with an electric panel providing charging ports for portable electronic devices, converters, transformers and surge protectors for a plurality of AC and DC receptacles as power source for the on-board equipment including one or more computers storing application specific software for the user interface.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A robotic device comprising:
an articulatable elongate member comprising a proximal end and a distal end, wherein the distal end is steerable via a first driving mechanism;
an articulatable imaging instrument removably coupled to the articulatable elongate member via a first lumen of the articulatable elongate member, wherein the articulatable imaging instrument comprises a camera located at a distal portion of the articulatable imaging instrument;
a first articulatable instrument removably coupled to the articulatable elongate member via a second lumen; and
a second articulatable instrument removably coupled to the articulatable elongate member via a third lumen, wherein an operation of the first articulatable instrument and the second articulatable instrument is captured by the camera of the articulatable imaging instrument.

2. The robotic device of claim 1, wherein an exit of the first lumen and an exit of the second lumen are located on substantially opposing sides of the distal end of the articulatable elongate member such that the first articulatable instrument, the second articulatable instrument and the camera are positioned to a triangulation configuration when the first articulatable instrument and the second articulatable instrument are performing the operation.

3. The robotic device of claim 1, wherein the articulatable elongate member comprises a bending section.

4. The robotic device of claim 3, wherein the bending section is articulated by one or more pull wires.

5. The robotic device of claim 1, wherein the articulatable imaging instrument comprises a bending section.

6. The robotic device of claim 5, wherein the bending section is articulated by one or more pull wires.

7. The robotic device of claim 1, wherein the articulatable imaging instrument comprises an illuminating device located at the distal portion of the articulatable imaging instrument.

8. The robotic device of claim 1, wherein the articulatable imaging instrument comprises one or more nozzles for clearing a view of the camera.

9. The robotic device of claim 1, wherein the camera is controlled to roll about a longitudinal axis of the articulatable elongate member or a longitudinal axis of the articulatable imaging instrument.

10. The robotic device of claim 1, wherein the camera is controlled to have an articulation movement relative to the articulatable elongate member.

11. The robotic device of claim 1, wherein the articulatable imaging instrument and the first articulatable instrument are withdrawn into the first lumen and the second lumen when the robotic device is in a first mode.

12. The robotic device of claim 11, wherein the articulatable imaging instrument and the first articulatable instrument are extended out of the distal end of the articulatable elongate member when the robotic device is in a second mode.

13. The robotic device of claim 1, wherein the articulatable imaging instrument is steerable via the first driving mechanism.

14. The robotic device of claim 1, wherein the first driving mechanism is mounted to a first robotic support system.

15. The robotic device of claim 14, wherein the first articulatable instrument is articulated via a second driving mechanism.

16. The robotic device of claim 15, wherein the second driving mechanism is mounted to a second robotic support system.

17. The robotic device of claim 16, wherein the first robotic support system and the second robotic support system are operatively coupled.

18. The robotic device of claim 1, wherein the proximal end of the articulatable elongate member is removably coupled to the first driving mechanism.

19. A method for a robotic device comprising:
providing an articulatable elongate member comprising a plurality of lumens;
coupling an articulatable imaging instrument to the articulatable elongate member via a first lumen of the plurality of lumens, wherein the articulatable imaging instrument comprises a camera located at a distal portion of the articulatable imaging instrument;
coupling a first articulatable instrument to the articulatable elongate member via a second lumen of the plurality of lumens;
coupling a second articulatable instrument to the articulatable elongate member via a third lumen of the plurality of lumens; and
capturing an operation of the first articulatable instrument and the second articulatable instrument by the camera of the articulatable imaging instrument.

20. The method of claim 19, wherein an exit of the first lumen and an exit of the second lumen are located on substantially opposing sides of the distal end of the articulatable elongate member such that the first articulatable instrument, the second articulatable instrument and the camera are positioned to a triangulation configuration when the first articulatable instrument and the second articulatable instrument are performing the operation.
